(19) United States
(12) Patent Application Publication
(10) Pub. No.: US 2018/0193520 A1
(43) Pub. Date: Jul. 12, 2018

(54) Injectable Polyurethanes and Applications Thereof

(71) Applicant: LifeCell Corporation, Branchburg, NJ (US)

(72) Inventor: Rick T. Owens, Stewartsville, NJ (US)

(21) Appl. No.: 15/850,872

(22) Filed: Dec. 21, 2017

Related U.S. Application Data
(60) Provisional application No. 62/438,615, filed on Dec. 23, 2016.

(51) Int. Cl. A61L 27/18; A61L 27/50; A61L 27/36

(52) U.S. Cl. CPC A61L 27/18; A61L 27/50; A61L 27/3608; A61L 2400/06; A61L 27/362; A61L 27/3612; A61L 27/3683; A61L 27/3633

(57) ABSTRACT

Polyurethane-based tissue fillers useful for treating and/or augmenting tissue, as well as acting as a biological scaffold that promotes cell in-ingrowth and tissue integration, are disclosed, as are quick-setting, injectable precursors of such tissue fillers. Such tissue fillers generally comprise (1) a polyurethane and (2) a particulate acellular tissue matrix. Also disclosed are methods of treating and/or augmenting tissues using such tissue fillers, particularly voids in human tissue such as anal fistulae or hernias.

This application claims priority under 35 USC § 119 to U.S. Provisional Application No. 62/438,615, which was filed Dec. 23, 2016 and is herein incorporated by reference in its entirety.

The present disclosure relates generally to polyurethane-based compositions that can be used treat to and/or augment tissue. The present disclosure also relates to methods of treating and/or augmenting tissues using such polyurethane-based compositions.

Treatment of voids or other defects in hard or soft tissue can present challenges due to their often irregular or even unknown geometries, such as in the repair of complex anal fistulae. Filler materials can be used for the treatment of such tissue and can conform to and set into the irregular or unknown geometries of such voids in vivo. These filler materials, however, should be able to resist migration, retain their volume and structural integrity over time, integrate well with surrounding tissue, and/or promote cell in-growth and tissue regeneration.

Semi-permanent and permanent injectable filler materials currently approved as aesthetic dermal fillers have been contemplated for use in the treatment of hard and soft tissue voids, particularly for the treatment of complex anal fistulae. However, many dermal fillers are responsible for both short- and long-term clinical complications that are product related. See de Vries, et al., Expert Review of Medical Devices, Vol. 10(6), pp. 835-53 (2013). For example, synthetic materials, such as cyanoacrylate glue, and biologically derived materials, such as BIOGLUE®, have been studied as biological infill materials in the treatment of anal fistulae. Lewis, et al., Colorectal Disease, Vol. 14, pp 1445-56 (2012). However, with regard to cyanoacrylate glue, histological data has shown that it acts as a barrier to host tissue integration, initiates a chronic inflammatory response, and can cause multiple abscess formation. Id. at 1447. Thus, the concern is that if used as an infill material, the glue will act in a palliative fashion by completely occluding the fistula until it starts degrading, resulting in either a recurrent fistula or acute sepsis. Id. Meanwhile, BIOGLUE® is associated with unacceptable rates of acute sepsis, often requiring surgical drainage, and may cause nerve injury, coagulation necrosis, and release glutaraldehyde levels that are toxic. Id. at 1448.

In contrast to the filler materials discussed above, polyurethane is highly biocompatible due to its chemical stability in physiological conditions, and accordingly, has found common use in the treatment of tissue (e.g., as wound dressings and adhesives). However, exposure to polyurethane has nonetheless been known to elicit an inflammatory response in certain patients. Furthermore, polyurethane lacks the ability to promote the degree of cell in-growth and tissue regeneration necessary for tissue void treatment. Accordingly, there exists a continued need for improved injectable filler materials that promote cell in-growth and tissue regeneration, while also being clinically safe.

The present disclosure provides for injectable, polyurethane-based filler materials that provide one or more of the aforementioned properties, as well as for methods of their use.

Thus, according to various embodiments, a composition comprising (1) a polyurethane precursor and (2) a particulate acellular tissue matrix is provided.

In certain embodiments, the above polyurethane precursor comprises at least one polyol. In certain other embodiments, the above polyurethane precursor comprises at least one polyamine.

In certain embodiments, the above composition further comprises at least one catalyst. In certain embodiments, the above composition further comprises one or more crosslinking agents, chain extending agents, blowing agents, surfactants, water-miscible solvents, water-binding compounds, or any combination thereof. In certain embodiments, the above composition further comprises water. In certain other embodiments, the above composition further comprises a poly(methylhydrosiloxane).

In certain embodiments, the above composition further comprises at least one polyisocyanate. In certain embodiments, the at least one polyisocyanate is a diisocyanate selected from the group consisting of toluene diisocyanate (TDI), methylene diphenyl diisocyanate (MDI), 1,6-hexamethylene diisocyanate (HDI), 1-isocyanato-3-isocyanatomethyl-3,5,5-trimethyl-cyclohexane (isophorone diisocyanate, IPDI), 4,4′-diisocyanato dicyclohexylmethane (H₁₂MDI), and combinations thereof. In certain embodiments, the at least one polyisocyanate is a polyisocyanate prepolymer. In certain other embodiments, the above compositions further comprise at least one polycyclic carbonate.

In certain embodiments, the particulate acellular tissue matrix of the above composition is derived from dermal tissue, adipose tissue, muscle tissue, bone tissue, cartilage tissue, or any combination thereof. In certain embodiments, the particulate acellular tissue matrix used to form the above composition is in the form of a slurry, a cryomilled dry powder, or micronized particles.

In certain embodiments, the weight ratio of polyurethane precursor to particulate acellular tissue matrix in the above composition is in the range of from 1:9 to 1:1.

In certain embodiments, the acellular tissue matrix of the above composition has been sterilized. In certain embodiments, the acellular tissue matrix has been sterilized via e-beam, gamma radiation, UV radiation, and/or supercritical CO₂.

According to other embodiments, a composition produced by polymerizing the at least one polyol with the at least one polyisocyanate of the above composition is provided. According to other embodiments, a composition produced by polymerizing the at least one polyamine with the at least one polycyclic carbonate of the above composition is provided. According to other embodiments, a composition produced by polymerizing the at least one polyamine with the at least one polyisocyanate prepolymer is provided. In certain embodiments, the acellular tissue matrix of each of the above compositions is derived from dermal tissue, adipose tissue, muscle tissue, bone tissue, cartilage tissue or any combination thereof.

According to other embodiments, a method of treating and/or augmenting tissue in a human or an animal comprising (a) providing a composition comprising (1) a polyurethane precursor comprising at least one polyol and (2) a particulate acellular tissue matrix, (b) providing at least one polyisocyanate, (c) mixing the composition of (a) and the at least one polyisocyanate of (b) to form a mixture and initiate polymerization of the at least one polyol and the at least one polyisocyanate, and (d) introducing the mixture of (c) into the tissue of a person or animal to be treated and/or augmented such that the polymerization of the at least one polyol and the at least one polyisocyanate is completed in situ is provided.

According to other embodiments, a method of treating and/or augmenting tissue in a human or an animal comprising (a) providing a composition comprising (1) a polyurethane precursor comprising at least one polyamine and (2) a particulate acellular tissue matrix, (b) providing at least one polycyclic carbonate, mixing the composition of (a) and the at least one polycyclic carbonate of (b) to form a mixture and initiate polymerization of the at least one polyamine and the at least one polycyclic carbonate, and (d) introducing the mixture of (c) into the tissue of a person or animal to be treated and/or augmented such that the polymerization of the at least one polyamine and the at least one polycyclic carbonate is completed in situ is provided.

According to other embodiments, a method of treating and/or augmenting tissue in a human or an animal comprising (a) providing a composition comprising (1) a polyurethane precursor comprising at least one polyamine and (2) a particulate acellular tissue matrix, (b) providing at least one polyisocyanate prepolymer, (c) mixing the composition of (a) and the at least one polyisocyanate prepolymer of (b) to form a mixture and initiate polymerization of the at least one polyamine and the at least one polyisocyanate prepolymer, and (d) introducing the mixture of (c) into the tissue of a person or animal to be treated and/or augmented such that the polymerization of the at least one polyamine and the at least one polyisocyanate prepolymer is completed in situ is provided.

In certain embodiments, the composition of (a) used in the above methods further comprises at least one catalyst. In certain embodiments, the composition of (a) used in the above methods further comprises one or more crosslinking agents, chain extending agents, surfactants, water-miscible solvents, water-binding compounds, or any combination thereof. In certain embodiments, the composition of (a) used in the above methods further comprises water. In certain other embodiments, the composition of (a) used in the above methods further comprises a poly(methylhydrosiloxane). In certain embodiments, the acellular tissue matrix used in the above methods is derived from dermal tissue, adipose tissue, muscle tissue, bone tissue, cartilage tissue, or any combination thereof. In certain embodiments, the mixture of (c) in the above methods is introduced into a void or defect in the tissue of the person or animal. In certain embodiments, that void or defect is an anal fistula or abdominal wall defect (e.g., hemia) in a human. In certain embodiments, the acellular tissue matrix used in the above methods has been sterilized prior to step (a). In certain embodiments, the acellular tissue matrix has been sterilized via e-beam, gamma radiation, UV radiation, and/or supercritical CO₂.

According to other embodiments, a kit comprising (1) a polyurethane precursor and (2) an acellular tissue matrix is provided. In certain embodiments, the kit further comprises (3) at least one catalyst. In certain embodiments, the kit further comprises (4) one or more crosslinking agents, chain extending agents, blowing agents, surfactants, water-miscible solvents, water-binding compounds, or any combination thereof. In certain embodiments, the kit further comprises a device capable of mixing components (1), (2), (3), and (4) and/or injecting a mixture of components (1), (2), (3), and (4). In certain embodiments, the device is selected from the group consisting of single barrel syringes, dual barrel syringe systems, cannulae, syringe-to-syringe luer lock adapter-based systems, in-line static mixers, mixing tips, or any combination thereof.

### BRIEF DESCRIPTION OF THE DRAWING

FIG. 1 depicts the open, scaffold-based structure that results from polymerization of a polyisocyanate prepolymer with a diamine chain extender and an embodiment of the present invention whereby the same polymerization in the presence of a particulate acellular tissue matrix results in entrapment of the tissue matrix particles within the polyurethane scaffold.

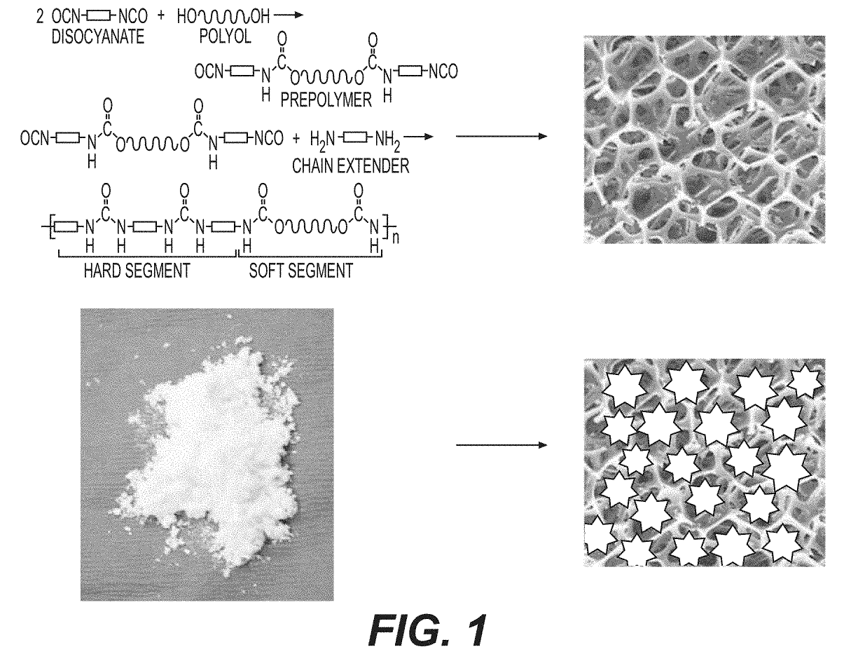

### DESCRIPTION OF EXEMPLARY EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any ranges described herein will be understood to include the endpoints and all values between the endpoints.

Any section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present disclosure provides for polyurethane-based filler materials. The materials can be injectable, quick-curing filler materials for use in the treatment and/or augmentation of voids in hard or soft tissue. The materials can act as biological scaffolds that conform to irregular and/or unknown three-dimensional geometries in vivo, stay in the desired location after implantation, retain their volume and structural integrity over time, integrate well with surrounding tissue, and/or promote cell in-growth and regeneration. Prior to polymerization, these disclosed filler materials are injectable precursor compositions that, at a minimum, comprise (1) a polyurethane precursor and (2) a particulate acellular tissue matrix.

The term “polyurethane precursor,” as used herein, refers generally to any polyol that can polymerize with a polyisocyanate to form a polyurethane, to any polyamine that can polymerize with (1) a poly(cyclic carbonate) to form a poly(β-hydroxyurethane), or (2) a polyurethane prepolymer to form a polyurethanepolyurea. The term “polyurethane,” as used herein, generally encompasses polyurethanes, poly(3-hydroxyurethanes), polyurethanepolyureas, and any other polymers or copolymers containing two or more urethane moieties in a backbone of the polymer chain. The term “polyol,” as used herein, refers generally to any organic compound substituted with at least two hydroxyl groups. The term “polyisocyanate,” as used herein, refers generally to any organic compound substituted with at least two isocyanate groups. The term “polyamine,” as used herein, refers generally to any organic compound substituted with at least two amino groups. The term “poly(cyclic carbonate),” as used herein, refers generally to any organic compound substituted with at least two cyclic carbonate groups having the following substructure:

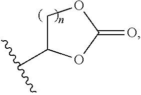

wherein n is 1 or 2. The term “polyisocyanate prepolymer,” as used herein, refers generally to any polyisocyanate polymer having at least two terminal isocyanate groups.

The polyurethane precursor of the presently disclosed precursor compositions may comprise any polyol suitable for forming the polyurethane component of the presently disclosed polyurethane-based filler materials. In certain embodiments, the at least one polyol can be a polyether polyol (e.g., a poly(oxyalkylene) polyol), a polyester polyol, a polyacrylate polyol, a polyurethane polyol, a polycarbonate polyol, a polycaprolactone polyol, a polybutadiene polyol, a polysulfide polyol, a polyether polyester polyol, a polyester polyacrylate polyol, a polyurethane polyacrylate polyol, a polyurethane polyester polyol, a polyurethane polyether polyol, a polyurethane polycarbonate polyol, a polyester polycarbonate polyol, or any combination thereof. In certain embodiments, such polymeric polyols have an average molecular weight in the range of from 62 to 8000. In certain embodiments, such polymeric polyols have an average molecular weight in the range of from 600 to 6000. In certain embodiments, such polymeric polyols have an average molecular weight in the range of from 800 to 4000. In certain embodiments, such polymeric polyols have an average hydroxyl functionality in the range of from 2 to 6. In certain embodiments, such polymeric polyols have an average hydroxyl functionality in the range of from 2.1 to 4. In certain embodiments, such polymeric polyols have an average hydroxyl functionality in the range of from 2.2 to 3. In certain embodiments, such polymeric polyols have an average hydroxyl functionality of 2.

In certain embodiments, the polyol can be a polyether polyol, which can be optionally mixed with other isocyanate reactive polymers, such as hydroxy-functional polybutadienes, polyester polyols, amino-terminated polyether polyols, and the like. Among the polyoxyalkylene polyols that can be used are the alkylene oxide adducts of a variety of suitable initiator molecules. Examples of such initiator molecules include, but are not limited to, dihydric initiators, such as ethylene glycol, diethylene glycol, triethylene glycol, propylene glycol, dipropylene glycol, butylene glycol, polyalkylene glycols, such as polyethylene glycol and polypropylene glycol, 1,3-butanediol, 1,4-butanediol, tripropylene glycol, neopentyl glycol, 1,3-propanediol, 1,4-butanediol, 1,6-hexanediol, 1,4-cyclo-hexanediol, 1,4-cyclohexanedimethanol, 1,5-pentenediol, 1,5-pentanediol, neopentyl glycol, 1,7-heptanediol, 1,8-octanediol, 1,10-decanediol, 2-methyl-1,3-propanediol, 2,2-dimethyl-1,3-propanediol, 3-methyl-1,5-pentanediol, 2-butyl-2-ethyl-1,3-propanediol, 2-butene-1,4-diol, 2-butyne-1,4-diol, tetraethylene glycol, dibutylene glycol, tributylene glycol, tetrabutylene glycol, dihexylene glycol, trihexylene glycol, tetrahexylene glycol and oligomer mixtures of alkylene glycols, such as diethylene glycol, hydroquinone, hydroquinone bis(2-hydroxy-ethyl)ether, the various bisphenols, such as bisphenol A, bisphenol F, and bis(hydroxyalkyl) ether derivatives thereof, aniline, the various N—N-bis(hydroxyalkyl)anilines, primary alkyl amines, and the various N—N-bis(hydroxyalkyl)amines; trihydric initiators, such as glycerine, trimethylolpropane, trimethylolbenzene, trishydroxyethyl isocyanurate, trimethylolethane, the various alkanolamines, such as ethanolamine, diethanolamine, triethanolamine, propanolamine, dipropanolamine, tripropanolamine, and polyethylene oxide polyols started on triols and having average molecular weights of from 62 g/mol to 400 g/mol; tetrahydric initiators, such as pentaerythritol, erythritol, sorbitan, ethylene diamine, N,N,N′N′-tetrakis[2-hydroxy-alkyl]ethylenediamines, toluene diamine, polyethylene oxide polyols started on tetraols and having average molecular weights of from 62 g/mol to 400 g/mol, and N,N,N′,N′-tetrakis[hydroxy-alkyl]toluene diamines; pentahydric initiators, such as the various alkylglucosides, (e.g., α-methylglucoside); hexahydric initiators, such as sorbitol, mannitol, hydroxyethylglucoside, and hydroxypropyl glucoside; octahydric initiators such as sucrose; higher functionality initiators, such as various starches and partially hydrolyzed starch-based products; and methylol group-containing resins and novolak resins, such as those prepared from the reaction of an aldehyde (e.g., formaldehyde) with a phenol, cresol, or other aromatic hydroxyl-containing compound, or any combination thereof. In certain embodiments, the polyol can be an ethylene oxide, propylene oxide, butylene oxide, and/or styrene oxide adduct of the above initiators, such as glycol, glycerine, pentaerythritol, trimethylolpropane, sorbitol, and sucrose; or any combination thereof. Processes for preparing such polyoxyalkylene polyols are well known in the art. The most common process for polymerizing such polyols is the base-catalyzed addition of the oxide monomers to the active hydrogen groups of the polyhydric initiator followed by subsequent addition to the oligomeric polyol moieties. Potassium hydroxide and sodium hydroxide are the most commonly used basic catalysts in this process. In certain other embodiments, the polyether polyol can be a polytetramethylene glycol polyether. Such polyether polyols can be prepared by polymerization of tetrahydrofuran via cationic ring opening.

In certain embodiments, the polyol can be a polyester polyol. Such polyester polyols include polycondensates of polyols and polycarboxylic acids, hydroxycarboxylic acids, and/or lactones. Instead of free polycarboxylic acids, their corresponding polycarboxylic anhydrides or corresponding polycarboxylic esters of lower alcohols can also be used to prepare such polyester polyols. Examples of suitable polyols include, but are not limited to, those listed above as initiator molecules for polyether polyols. Examples of suitable polycarboxylic acids include, but are not limited to phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, succinic acid, 2-methylsuccinic acid, 3,3-diethylglutaric acid, 2,2-dimethylsuccinic acid, dodecanedioic acid, decanedicarboxylic acid, pimelic acid, sebacic acid, isoterephthalic acid, endomethylenetetrahydrophthalic acid, dimer fatty acid, trimer fatty acid, citric acid, trimellitic acid, or any combination thereof. The corresponding anhydrides, and esters and hemiesters of low molecular weight monohydric alcohols having from 1 to 4 carbon atoms of these acids can also be used as the acid source. To the extent that the average functionality of the polyol to be esterified is ≥2, it is also possible to make additional concomitant use of monocarboxylic acids such as benzoic acid and hexanecarboxylic acid. Examples of suitable hydroxycarboxylic acids include, but are not limited to, hydroxycaproic acid, hydroxybutyric acid, hydroxydecanoic acid, hydroxystearic acid, or any combination thereof. Examples of suitable lactones include, but are not limited to, caprolactone, butyrolactone, homologs thereof, or any combination thereof.

In certain embodiments, the polyol can be a polycarbonate polyols. Such polyols are obtainable through reaction of carbonic acid derivatives, such as diphenyl carbonate, dimethyl carbonate, or phosgene, with polyols, such as diols, or through the copolymerization of alkylene oxides, such as propylene oxide, with CO₂. Examples of suitable diols include, but are not limited to, ethylene glycol, 1,2-propanediol, 1,3-propanediol, 1,3-butanediol, 1,4-butanediol, 1,6-hexanediol, 1,8-octanediol, neopentyl glycol, 1,4-bishydroxy-methylcyclohexane, 2-methyl-1,3-propanediol, 2,2,4-trimethyl-1,3-pentanediol, dipropylene glycol, polypropylene glycols, dibutylene glycol, polybutylene glycols, bisphenol A, or any combination thereof. Instead of or in addition to pure polycarbonate polyols, polyether polycarbonate diols can also be prepared and used.

In certain embodiments, the polyol can be a polyacrylate polyol. Such polyols are obtainable through free-radical polymerization of hydroxyl-containing olefinically unsaturated monomers or through free-radical copolymerization of hydroxyl-containing olefinically unsaturated monomers with optionally other olefinically unsaturated monomers. Examples of suitable hydroxyl-containing olefinically unsaturated monomers include, but are not limited to, 2-hydroxyethyl acrylate, 2-hydroxyethyl methacrylate, the hydroxypropyl acrylate isomer mixture obtainable through addition of propylene oxide onto acrylic acid, the hydroxypropyl methacrylate isomer mixture obtainable through addition of propylene oxide onto methacrylic acid, or any combination thereof. Examples of suitable olefinically unsaturated monomers include, but are not limited to, ethyl acrylate, butyl acrylate, 2-ethylhexyl acrylate, isobornyl acrylate, methyl methacrylate, ethyl methacrylate, butyl methacrylate, cyclohexyl methacrylate, isobornyl methacrylate, styrene, acrylic acid, acrylonitrile, methacrylonitrile, or any combination thereof. Examples of suitable free-radical initiators for use in polymerizing such monomers include, but are not limited to, azo compounds, e.g., azoisobutyronitrile (AIBN) and peroxides, e.g., di-tert-butyl peroxide.

In certain embodiments, the polyol can be a polyether polyester polyol. Such polymers contain ether groups, ester groups, and hydroxyl groups. Suitable compounds for producing such polyether polyester polyols are polycarboxylic acids, such as aliphatic dicarboxylic acids and/or aromatic dicarboxylic acids, having up to 12 carbon atoms and polyether polyols obtained through alkoxylation of initiator molecules, such as polyhydric alcohols. Examples of suitable polycarboxylic acids include, but are not limited to, any one or combination of those listed above for polyester polyols. The initiator molecules used to prepared the polyether polyols are at least difunctional, but can also optionally contain proportions of starter molecules of higher functionality, especially trifunctional starter molecules. Examples of suitable initiator molecules include, but are not limited to, any one or combination of those listed above for polyether polyols. Polyether polyester polyols can also be prepared by alkoxylation of reaction products which are obtained by the reaction of polycarboxylic acids and diols.

The polyurethane precursor of the presently disclosed precursor compositions can comprise at least one polyamine suitable for forming the polyurethane component of the presently disclosed polyurethane-based filler materials. Examples of such polyamines include, but are not limited to, ethylenediamine, 1,2-diaminopropane, 1,4-diaminobutane, 1,3-diaminopentane, 1,5-diaminopentane, 2-methyl-1,5-diaminopentane, 1,6-diaminohexane, 2,5-diamino-2,5-dimethylhexane, 2,2,4-trimethyl-1,6-diaminohexane, 2,4,4-trimethyl-1,6-diaminohexane, 1,11-diaminoundecane, 1,12-diaminododecane, 1-amino-3,3,5-trimethyl-5-aminomethylcyclohexane, 2,4-hexahydrotolylenediamine, 2,6-hexahydrotolylenediamine, 2,4′-diaminodicyclohexylmethane, 4,4′-diaminodicyclohexylmethane, 3,3′-dimethyl-4,4′-diaminodicyclohexylmethane, 2,4,4′-triamino-5-methyldicyclohexylmethane, lysine, polyetheramines having aliphatically attached primary amino groups and a number-average molecular weight Mₙ in the range of from 148 to 6000 g/mol, the corresponding polyamines resulting from complete hydrolysis of the isocyanate groups of the polyisocyanates disclosed herein with water, or any combination thereof.

In certain embodiments, the presently disclosed precursor compositions can further comprise a polyurethane catalyst. Suitable polyurethane catalysts are well known in the art, an extensive list of which is provided in U.S. Pat. No. 5,011,908, which is incorporated herein by reference. Classes of the most commonly used polyurethane catalysts include tertiary amines and organotin compounds. Examples of suitable tertiary amine polyurethane catalysts include, but are not limited to, trimethylamine, triethylamine, dimethylcyclohexylamine, N-methylmorpholine, N,N′-dimethylpiperazine, 2-(dimethylaminoethoxy)ethanol, diazabicyclo-[2.2.2]octane, triethylenediamine, bis(2,2′-dimethylamino)ethyl ether, N-ethylmorpholine, and diethylenetriamine. Examples of suitable organotin polyurethane catalysts include, but are not limited to, stannous diacetate, stannous dioctoate, stannous oleate, stannous dilaurate, dibutyltin diacetate, dibutyltin dilaurate. In certain embodiments, the polyurethane catalyst can be present in the precursor composition in an amount in the range of from 0.001 to 2 parts per 100 parts of polyol and/or polyamine. In certain embodiments, the polyurethane catalyst can be present in the precursor composition in an amount in the range of from 0.05 to 1 part per 100 parts of polyol and/or polyamine.

In certain embodiments, the presently disclosed precursor compositions can further comprise a chain extender or crosslinker. In certain embodiments, suitable chain extenders and crosslinkers can be any aliphatic, araliphatic, aromatic, or cycloaliphatic polyol, polyamine, and/or aminoalcohol. In certain embodiments, such compounds have a molar mass in the range of from 50 to 499 and from 2 to 10 carbon atoms. Examples of suitable chain extenders include, but are not limited to, ethylene glycol, propylene glycol, diethylene glycol, dipropylene glycol, 2-methyl-1,3-propanediol, ethylene diamine, 1,4-butanediol, 1,3-butanediol, 2,3-butanediol, 1,2-butanediol, 1,5-pentanediol, 1,6-hexanediol, ethanolamine, and polyethylene glycols having a weight-average molecular weight of up to 200. Examples of suitable crosslinkers include, but are not limited to, polyols and alkanolamines, such as trimethylolpropane, glycerine, sorbitol, diethanolamine, triethanolamine, or any combination thereof. In certain embodiments, a chain extender or crosslinker is included in the precursor composition in an amount in the range of from 0.1 to 5 weight %, based on the amount of isocyanate-reactive mixture.

Pores can be formed in the presently disclosed polyurethane-based filler material during polymerization by use of a blowing agent. Thus, in certain embodiments, the presently disclosed precursor compositions can further comprise a blowing agent. Such blowing agents can be in the form of a gas or liquid. Examples of such gases include, but are not limited to, carbon dioxide, nitrogen, argon, or air. Examples of such liquids include, but are not limited to, water, low boiling, polyhalogenated organic compounds, and poly(methylhydrosiloxanes). In certain embodiments, the gaseous or liquid blowing agents can diffuse out of the cured filler material, thereby providing pores for biological in-growth.

In certain embodiments, the presently disclosed precursor compositions can further comprise a surfactant. Surfactants can be added to the presently disclosed precursor compositions to, for example, disperse prepolymers, polyols, and other additional components, stabilize carbon dioxide bubbles, and/or control pore sizes of the resulting polyurethane-based filler materials. Such surfactants can include non-ionic surfactants, anionic surfactants, cationic surfactants, or any combination thereof. In certain embodiments, the surfactant is non-toxic at the concentration in which it remains in the polyurethane. Examples of such surfactants include, but are not limited to, polyethersiloxanes, salts of fatty sulfonic acids, salts of fatty acids, or any combination thereof. In certain embodiments, the surfactant is a polyethersiloxane and its concentration in the precursor composition can, for example, be in the range of from 0.25 to 4 parts per hundred of polyol or polyamine. In some embodiments, the polyethersiloxane is hydrolyzable. In certain other embodiments, the surfactant can be a salt of a fatty sulfonic acid and its concentration in the precursor composition can, for example, be in the range of approximately 0.5 to 5 parts per hundred of polyol or polyamine. Examples of suitable salts of fatty sulfonic acids include, but are not limited to, sulfated castor oil or sodium ricinoleicsulfonate.

In certain embodiments, the presently disclosed precursor compositions can further comprise a water-miscible solvent. In certain embodiments, those water-miscible solvents are biocompatible water-miscible solvents that are chemically inert to the polyisocyanates, poly(cyclic carbonates), and polyisocyanate prepolymers used to form the presently disclosed polyurethane-based tissue fillers. Examples of suitable biocompatible and chemically inert water miscible solvents include, but are not limited to, dioxane, dimethylformamide, N-methylpyrollidone, and dimethylsulfoxide.

In certain embodiments, the presently disclosed precursor compositions can further comprise a water-binding compound. An example of a suitable water-binding compound includes, but is not limited to, hyaluronic acid.

The term “acellular tissue matrix,” as used herein, refers generally to any tissue matrix that is substantially free of cells and/or cellular components. The acellular tissue matrices of the presently disclosed compositions may be derived from any type of tissue. Examples of the tissues that may be used to construct the acellular tissue matrices of the presently disclosed precursor compositions include, but are not limited to, skin (i.e., dermal), parts of skin (e.g., dermis), adipose, fascia, muscle (striated, smooth, or cardiac), pericardial tissue, dura, umbilical cord tissue, placental tissue, cardiac valve tissue, ligament tissue, tendon tissue, blood vessel tissue, such as arterial and venous tissue, cartilage, bone, neural connective tissue, urinary bladder tissue, ureter tissue, and intestinal tissue. In certain embodiments, the tissue matrices used according to the present invention can comprises cells. An example of such a cellular tissue matrix includes, but is not limited to, autologous fat tissue.

The acellular tissue matrices of the presently disclosed compositions can be selected to provide a variety of different biological and/or mechanical properties. For example, an acellular tissue matrix can be selected to allow tissue in-growth and remodeling to assist in regeneration of tissue normally found at the site where the matrix is implanted. In certain embodiments, the acellular tissue matrices of the present disclosure can be selected or derived from ALLODERM® or STRATTICET (LIFECELL CORPORATION, Branchburg, N.J.), which are human and porcine acellular dermal matrices, respectively. In certain other embodiments, the particulate acellular tissue matrix can include a cryofractured tissue matrix material, such as CYMETRA®, (LifeCell Corporation, Branchburg, N.J.), which is an injectable form of ALLODERM®. In certain other embodiments, the acellular tissue matrix can include demineralized bone matrix (i.e., DBM). Alternatively, other suitable acellular tissue matrices can be used, as described further below.

Tissue matrices can be processed in a variety of ways to produce decellularized (i.e., acellular) tissue matrices. In general, the steps involved in the production of an acellular tissue matrix include harvesting the tissue from a donor (e.g., a human cadaver or animal source) and cell removal under conditions that preserve biological and structural function. In certain embodiments, the process includes chemical treatment to stabilize the tissue and avoid biochemical and structural degradation together with or before cell removal. In various embodiments, the stabilizing solution arrests and prevents osmotic, hypoxic, autolytic, and proteolytic degradation, protects against microbial contamination, and reduces mechanical damage that can occur with tissues that contain, for example, smooth muscle components (e.g., blood vessels). The stabilizing solution may contain an appropriate buffer, one or more antioxidants, one or more oncotic agents, one or more antibiotics, one or more protease inhibitors, and/or one or more smooth muscle relaxants.

The tissue is then placed in a decellularization solution to remove viable cells (e.g., epithelial cells, endothelial cells, smooth muscle cells, and fibroblasts) from the structural matrix without damaging its biological and structural integrity. The decellularization solution may contain an appropriate buffer, salt, an antibiotic, one or more detergents, one or more agents to prevent crosslinking, one or more protease inhibitors, and/or one or more enzymes.

Acellular tissue matrices can be tested or evaluated to determine if they are substantially free of cell and/or cellular components in a number of ways. For example, processed tissues can be inspected with light microscopy to determine if cells (live or dead) and/or cellular components remain. In addition, certain assays can be used to identify the presence of cells or cellular components. For example, DNA or other nucleic acid assays can be used to quantify remaining nuclear materials within the tissue matrices. Generally, the absence of remaining DNA or other nucleic acids will be indicative of complete decellularization (i.e., removal of cells and/or cellular components). Finally, other assays that identify cell-specific components (e.g., surface antigens) can be used to determine if the tissue matrices are acellular. Finally, other assays that identify cell-specific components (e.g., surface antigens) can be used to determine if the tissue matrices are acellular. After the decellularization process, the tissue sample is washed thoroughly with saline.

While an acellular tissue matrix may be made from one or more individuals of the same species as the recipient of the acellular tissue matrix, this need not necessarily be the case. Thus, for example, an acellular tissue matrix may be made from porcine tissue and implanted in a human patient. Species that can serve as recipients of acellular tissue matrix and donors of tissues or organs for the production of the acellular tissue matrix include, without limitation, mammals, such as humans, nonhuman primates (e.g., monkeys, baboons, or chimpanzees), pigs, cows, horses, goats, sheep, dogs, cats, rabbits, guinea pigs, gerbils, hamsters, rats, or mice.

Elimination of the α-gal epitopes from the collagen-containing material may diminish the immune response against the collagen-containing material. The α-gal epitope is expressed in non-primate mammals and in New World monkeys (monkeys of South America) as well as on macromolecules such as proteoglycans of the extracellular components. U. Galili et al., J. Biol. Chem., 263: 17755 (1988). This epitope is absent in Old World primates (monkeys of Asia and Africa and apes) and humans, however. Id. Anti-gal antibodies are produced in humans and primates as a result of an immune response to α-gal epitope carbohydrate structures on gastrointestinal bacteria. U. Galili et al., Infect. Immun., 56:1730 (1988); R. M. Hamadeh et al., J. Clin. Invest., 89:1223 (1992).

Accordingly, in certain embodiments, when animals that produce α-gal epitopes are used as the tissue source, the substantial elimination of α-gal epitopes from cells and from extracellular components of the acellular tissue matrix, and the prevention of re-expression of cellular α-gal epitopes can diminish the immune response against the acellular tissue matrix associated with anti-gal antibody binding to α-gal epitopes.

To remove α-gal epitopes, the tissue sample may be subjected to one or more enzymatic treatments to remove certain immunogenic antigens, if present in the sample. In some embodiments, the tissue sample may be treated with an α-galactosidase enzyme to eliminate α-gal epitopes, if present in the tissue. Any suitable enzyme concentration and buffer can be used as long as sufficient removal of antigens is achieved.

Alternatively, rather than treating the tissue with enzymes, animals that have been genetically modified to lack one or more antigenic epitopes may be selected as the tissue source. For example, animals (e.g., pigs) that have been genetically engineered to lack the terminal α-galactose moiety can be selected as the tissue source. For descriptions of appropriate animals see U.S. Patent Application Pub. No. 2005/0028228 A1 and U.S. Pat. No. 6,166,288, the disclosures of which are incorporated herein by reference in their entirety. In addition, certain exemplary methods of processing tissues to produce acellular tissue matrices with or without reduced amounts of or lacking alpha-1,3-galactose moieties, are described in Xu, Hui et al., “A Porcine-Derived Acellular Dermal Scaffold that Supports Soft Tissue Regeneration: Removal of Terminal Galactose-α-(1,3)-Galactose and Retention of Matrix Structure,” Tissue Engineering, Vol. 15, 1-13 (2009), which is incorporated by reference in its entirety.

In certain embodiments, the acellular tissue matrix can be sterilized prior to use. Sterilization of the acellular tissue matrix can be achieved by any suitable means known in the art. Examples of such means include, but are not limited to, sterilization via e-beam, gamma radiation, UV radiation, and/or supercritical CO₂.

The following procedure can be used to produce particulate acellular tissue matrices using ALLODERM®, STRATTICE™, or other suitable acellular tissue matrices (ATM). After removal from the packaging, acellular tissue matrix can be cut into strips using a Zimmer mesher fitted with a non-interrupting “continuous” cutting wheel. The resulting long strips of ATM may be cut into lengths of about 1 to about 2 centimeters in length.

A homogenizer and sterilized homogenizer probe, such as a LabTeck Macro homogenizer available from OMNI International, Warrenton, Va., may be assembled and cooled to cryogenic temperatures using sterile liquid nitrogen that is poured into the homogenizer tower. Once the homogenizer has reached cryogenic temperatures, acellular tissue matrix previously prepared into strips, as noted above, can be added to the homogenizing tower containing sterile liquid nitrogen. The homogenizer may then be activated so as to cryogenically fracture the strips of acellular tissue matrix. The time and duration of the cryogenic fractionation step will depend upon the homogenizer utilized, the size of the homogenizing chamber, the speed and time at which the homogenizer is operated, and should be able to be determined by one of skill in the art by simple variation of the parameters to achieve the desired results.

In certain embodiments where the acellular tissue matrix is a cryofractured tissue matrix material, such as CYMETRA®, a cryomilling process using a Spex Freezer mill is employed to manufacture the particulate acellular tissue matrix. Particulate acellular tissue matrix can also be manufactured via a dry grinding process using a Retsch SM300. Wet grinding techniques to manufacture the particulate acellular tissue matrix using a Stephan MC15 Rotor Stator can also be employed.

The cryofractured particulate acellular tissue matrix material may be sorted by particle size by washing the product of the homogenizer with liquid nitrogen through a series of metal screens that have also been cooled to liquid nitrogen temperatures. A combination of screens may be utilized within the homogenizing tower of the type described above in which the particles are washed and sorted first to exclude oversized particles and then to exclude undersized particles.

Once isolated, the particulate acellular tissue matrix may be removed and placed in a vial for freeze drying once the sterile liquid nitrogen has evaporated. This may ensure that any residual moisture that may have been absorbed during the above procedure is removed.

The final product can be a powder having any particle size suitable for injection. In certain embodiments, the acellular tissue matrix can have a particle size in the range of about 0.01 microns to 900 microns, 1 micron to about 900 microns or a particle size in the range of about 30 microns to about 750 microns. The particles are distributed about a mean of about 150-300 microns. In certain embodiments, the viscosity of the presently disclosed precursor compositions is such that it can pass through a 27 G needle or smaller bore needle. In certain embodiments, the particle size of the acellular tissue matrices of such precursor compositions is 250 microns or less. In certain embodiments, the particle size of the acellular tissue matrices of such precursor compositions is less than 1 micron. In certain embodiments, the particle size of the acellular tissue matrices of such precursor compositions is about 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 105 μM, 110 μM, 115 μM, 120 μM, 125 μM, 130 μM, 135 μM, 140 μM, 145 μM, 150 μM, 155 μM, 160 μM, 165M, 170 μM, 175 μM, 180 μM, 185 μM, 190 μM, 195 μM, 200 μM, 205 μM, 210 μM, 2155 μM, 220 μM, 225 μM, 230 μM, 235 μM, 240 μM, 245 μM, or 250 μM. In certain other embodiments, the particle size of the acellular tissue matrices of such precursor compositions is greater than 250 microns. The material is readily rehydrated by suspension in normal saline or other similar suitable rehydrating agent. The rehydrated acellular tissue matrix may be resuspended in normal saline or any other suitable pharmaceutically compatible carrier.

In certain embodiments, the presently disclosed precursor compositions can be prepared by thoroughly physically mixing a polyurethane precursor component comprising at least one polyol and/or at least one polyamine with the particulate acellular tissue matrix component. In certain embodiments, any combination of at least one catalyst, crosslinking agent, chain extending agent, blowing agent, surfactant, water-miscible solvent, and/or water-binding compound can also be thoroughly physically mixed with the polyurethane precursor and particulate acellular tissue components of the presently disclosed precursor compositions. The particulate acellular tissues matrices can be in any suitable physical form that does not interfere with, and preferably facilitates, homogeneous mixing with the polyurethane precursor and any other components that can be included in the presently disclosed precursor composition. Examples of such physical forms for the acellular tissue matrix include, but are not limited to, cryomilled dry powders, micronized particles, and non-aqueous slurries thereof. These components can be mixed by any means known in the art. When mixed together, the combination of the polyurethane precursor and particulate acellular tissue matrix components, as well as any crosslinking agents, chain extending agents, blowing agents, surfactants, water-miscible solvents, and water-binding compounds, form the presently disclosed precursor compositions, which can be in any suitable physical form that does not interfere with, and preferably facilitates, polymerization of the polyol and/or polyamine with the polyisocyanate, polycyclic carbonate, and/or polyisocyanate prepolymer. Examples of such suitable physical forms include, but are not limited to, solids, such as powders and granules of any particle size, and liquids, such as solutions, suspensions, dispersions, emulsions, or any combination thereof. In certain embodiments, the primary liquid medium of these solutions, suspensions, dispersions, and/or emulsions is the at least one polyol and/or the at least one polyamine. In certain embodiments, the primary liquid medium of these solutions, suspensions, dispersions, and/or emulsions is a water-miscible solvent.

The polyurethane precursor can be present in the precursor composition in any suitable concentration.

The polyurethane precursor and the particulate acellular tissue matrix can be present in the presently disclosed precursor compositions in any suitable weight ratio to each other. In certain embodiments, the weight ratio of polyurethane precursor and the particulate acellular tissue matrix in the presently disclosed precursor compositions is in the range of from 1:9 to 1:1.

In certain embodiments, the presently disclosed precursor compositions can be combined with at least one polyisocyanate and polymerized with the polyurethane precursor (i.e., comprising at least one polyol) to form the presently disclosed polyurethane-based filler materials. Such polyisocyanates can include any suitable aliphatic, cycloaliphatic, araliphatic, and/or aromatic polyisocyanate capable of polymerizing with a polyol to form a polyurethane. Examples of such polyisocyanates include, but are not limited to, trimethylene diisocyanate, tetramethylene diisocyanate, pentamethylene diisocyanate, hexamethylene diisocyanate (i.e., HDI), heptamethylene diisocyanate, octamethylene diisocyanate, 2-methylpentamethylene 1,5-diisocyanate, 2-ethylbutylene 1,4-diisocyanate, pentamethylene 1,5-diisocyanate, butylene 1,4-diisocyanate, 1-isocyanato-3,3,5-trimethyl-5-isocyanatomethylcyclohexane (i.e., isophorone diisocyanate, IPDI), 1,4- and 1,3-bis(isocyanatomethyl)cyclohexane (HXDI), cyclohexane 1,4-diisocyanate, 1-methylcyclohexane 2,4- and 2,6-diisocyanate, dicyclohexylmethane 4,4′-, 2,4′-, and 2,2′-diisocyanate, diphenylmethane 2,2′-, 2,4′-, and 4,4′-diisocyanate (i.e., MDI), naphthylene 1,5-diisocyanate (i.e., NDI), tolylene 2,4- and 2,6-diisocyanate (i.e., TDI), diphenylmethane diisocyanate, 3,3′-dimethylbiphenyl diisocyanate, 1,2-diphenylethane diisocyanate, phenylene diisocyanate, 4,4′-diisocyanato dicyclohexylmethane (H₁₂MDI), 2,2,4- and 2,4,4-trimethylhexamethylene diisocyanate, the isomeric bis(4,4′-isocyanatocyclohexyl)methanes, 1,4-phenylene diisocyanate, polymeric MDI, 1,3- and 1,4-bis(2-isocyanatoprop-2-yl)benzene (TMXDI), 1,3-bis-(isocyanatomethyl)benzene (XDI), alkyl 2,6-diisocyanatohexanoates (i.e., lysine diisocyanates) having C₁ to C₆ alkyl groups, 4,4′-dicyclohexylmethane diisocyanate (HuMDI), 2,2,4-(2,2,4)-trimethylhexamethylene diisocyanate (TMDI), dimers and trimers prepared from these polyisocyanates, or any mixture thereof.

In certain embodiments, the presently disclosed precursor compositions can be combined with at least one poly(cyclic carbonate) and polymerized with the polyurethane precursor (i.e., comprising at least one polyamine) to form the presently disclosed polyurethane-based filler materials. Such poly(cyclic carbonates) can include any suitable poly(cyclic carbonate) capable of polymerizing with a polyamine to form a β-hydroxypolyurethane. In certain embodiments, such poly(cyclic carbonates) can be prepared by reacting a polyisocyanate or polyisocyanate prepolymer, as provided herein, with a cyclic hydroxyalkyl carbonate having a 5- or 6-membered ring, such as glycerine carbonate, as disclosed in U.S. Pat. No. 8,118,968 B2, which is incorporated herein in its entirety. In certain embodiments, the use of poly(cyclic carbonates) to prepare the presently disclosed polyurethane-based tissue fillers can be a more biocompatible alternative to potentially toxic polyisocyanates or polyisocyanate prepolymers.

In certain embodiments, the presently disclosed precursor compositions can be combined with at least one polyisocyanate prepolymer and polymerized with the polyurethane precursor (i.e., comprising at least one polyol and/or at least one polyamine) of the precursor composition to form the presently disclosed polyurethane-based filler materials. In certain embodiments, polyurethane prepolymers can be prepared by polymerizing at least one polyol, as described above, with an excess of at least one polyisocyanate, as described above. Thus, the resulting polyisocyanate prepolymer is a polyurethane having terminal isocyanate groups solubilized in an excess of polyisocyanates. In certain other embodiments, the polyisocyanate prepolymer can be formed by using an approximately stoichiometric amount of polyisocyanates in forming a prepolymer and subsequently adding additional polyisocyanates.

A homogenous polyurethane reaction mixture can be prepared by thoroughly physically mixing the presently disclosed precursor composition with at least one polyisocyanate, at least one poly(cyclic carbonate), or at least one polyisocyanate prepolymer. Once combined, this homogenous polyurethane reaction mixture can be in any suitable form for injection in vivo. In certain embodiments, the homogenous polyurethane reaction mixture is in the form of a liquid. In certain embodiments, this liquid is in the form of a solution, a suspension, a dispersion, an emulsion or any combination thereof. In certain embodiments, the medium for such solutions, suspensions, dispersions, and emulsions is (1) the polyol and/or polyamine, (2) the at least one polyisocyanate, at least one poly(cyclic carbonate), or at least one polyisocyanate prepolymer, (3) at least one water-miscible solvent, or (4) any combination thereof. The presently homogenous polyurethane reaction mixtures can have any viscosity suitable for injection in vivo. To the extent that the viscosity of the mixture of the presently disclosed precursor compositions with the at least one polyisocyanate, at least one poly(cyclic carbonate), or at least one polyisocyanate prepolymer is not suitable or optimal for injection in vivo (i.e., the mixture is too viscous). In certain embodiments, at least one water-miscible solvent can be added to the mixture to modulate its viscosity lower.

The at least one polyol and/or the at least one polyamine of the polyurethane precursor and the at least one polyisocyanate, at least one poly(cyclic carbonate), or at least one polyisocyanate prepolymer can be combined in any suitable ratio of isocyanate reactive groups (i.e., hydroxyl and amino groups of the polyols and polyamines, respectively) or cyclic carbonate reactive groups (i.e., amino groups of the polyamines) to isocyanate groups (i.e., of the polyisocyanates and polyisocyanate prepolymers) or cyclic carbonate groups (i.e., the poly(cyclic carbonates)). In certain embodiments, the ratio of isocyanate or cyclic carbonate reactive groups to isocyanate or cyclic carbonate groups, respectively, is stoichiometric or substantially stoichiometric, such that there are no or substantially no unreacted isocyanate or cyclic carbonate reactive groups, isocyanate groups, or cyclic carbonate groups remaining after polymerization is complete. In certain other embodiments, the ratio of isocyanate or cyclic carbonate reactive groups to isocyanate or cyclic carbonate groups, respectively, is such that the isocyanate or cyclic carbonate reactive groups are in stoichiometric excess, such that there are no or substantially no unreacted isocyanate or cyclic carbonate groups remaining after polymerization is complete. In certain other embodiments, the ratio of isocyanate or cyclic carbonate reactive groups to isocyanate or cyclic carbonate groups, respectively, is such that the isocyanate or cyclic carbonate groups are in stoichiometric excess, such that there are no or substantially no unreacted isocyanate or cyclic carbonate reactive groups remaining after polymerization is complete.

The injectable, homogenous polyurethane reaction mixture can be administered to a human or an animal to treat and/or augment tissue. Thus, in certain embodiments, the method of administration comprises providing a first composition comprising (1) the presently disclosed polyurethane precursor comprising a polyol and/or a polyamine and (2) the presently disclosed particulate acellular tissue matrix, while separately providing a second composition comprising at least one polyisocyanate, at least one poly(cyclic carbonate), or at least one polyisocyanate prepolymer. In certain embodiments, the first composition optionally also comprises one or more crosslinking agents, chain extending agents, blowing agents, surfactants, water-miscible solvents, water-binding compounds, or any combination thereof. The first and second aqueous compositions are then mixed to initiate polymerization of the polyol and/or polyamine with the at least one polyisocyanate, at least one poly(cyclic carbonate), or at least one polyisocyanate prepolymer. This mixture is then introduced into the tissue of the person or animal to be treated and/or augmented, where the polymerization to form the presently disclosed polyurethane-based tissue filler is completed in situ. In certain embodiments, the first composition of this method comprises at least one polyol and the second composition comprises at least one polyisocyanate. In certain embodiments, the first composition comprises at least one polyamine and the second composition comprises at least one pol(cyclic carbonate). In certain embodiments, the first composition comprises at least one polyol and/or at least one polyamine and the second composition comprises at least one polyisocyanate prepolymer. In certain embodiments, the tissue comprises a void or defect, such as an anal fistula or abdominal wall defect (e.g., hemias including but not limited to inguinal hernias), to be filled and the mixture is then introduced into the void in the tissue of the person or animal such that the void is partially or completely filled and the polymerization is completed in situ.

The presently disclosed injectable, homogeneous polyurethane reaction mixture can be administered to a human or an animal by any suitable means known in the art. Examples of such means include, but are not limited to, via injection, via catheter, via cannula, or in bulk. Examples of such means of injection include, but are not limited to, single barrel syringes and dual barrel syringe systems that employ an in-line static mixer. In certain embodiments where a single barrel syringe is used, the first and second compositions can be mixed prior to loading into the barrel of the syringe, followed by subsequent injection into the situs to be treated and/or augmented. In certain embodiments where a dual barrel syringe system is used, the first composition can be loaded into one barrel of the dual barrel syringe, while the second composition can be loaded into the other barrel. Each composition is then simultaneously injected into the situs to be treated and/or augmented via the in-line static mixer, where the two compositions are homogeneously mixed before injection into the situs.

The polymerization to form the presently disclosed polyurethane-based tissue filler can be carried out under ordinary in vivo or metabolic conditions, i.e., temperatures in the range of from 35 to 39° C. and pH in the range of 6 to 7 (e.g., about 6.5). Thus, the polymerization can be performed in vivo to provide a polyurethane at a surgical situs to promote maximum seamless integration between the polymer and native tissue. In certain embodiments, integration of the polyurethane scaffold with native tissue can occur immediately as the polyisocyanate, poly(cyclic carbonate), or polyisocyanate prepolymer quickly penetrate into the existing tissue matrix prior to polymerization, and reacts not only with the polyol and/or polyamine of the polyurethane precursor, but also reacts and/or crosslinks with hydroxyl- and amino-group containing residues of resident proteins in the native tissue matrix. As such, the presently disclosed polyurethane-based tissue filler intrinsically possesses adhesive properties that allow it to adhere to adjacent tissue and prevent migration of the polymerized polyurethane/tissue matrix implant. This adhesive property also mitigates a typical problem found with pre-formed matrix plugs, which is their poor integration into native tissue. The ability to covalently integrate the polyurethane directly onto the tissue surface eliminates the need to surgically enlarge a defect to fit a pre-cast plug, as is necessary for tissue fillers whose chemistries are toxic to or otherwise prohibit their formation inside the patient.

In certain embodiments, the chemical structure of the polyurethane of the presently disclosed polyurethane-based tissue fillers can be varied to modulate its tissue adhesion properties, resorption profile, stiffness, and the shape of its three-dimensional structure.

As disclosed herein, the presently disclosed polyurethane-based tissue fillers intrinsically possess tissue adhesion properties. Thus, in certain embodiments, the adhesiveness of the presently disclosed polyurethane-based tissue fillers to native tissue can be enhanced or decreased by modulation of the amount of available isocyanate or cyclic carbonate groups that can react with the hydroxyl- and amino-group containing residues of resident proteins in the native tissue matrix. In other words, the adhesive properties can be increased or decreased by providing a stoichiometric excess or shortage, respectively, of isocyanate or cyclic carbonate groups relative to isocyanate or cyclic carbonate reactive groups in the polyurethane reactive mixture. In certain embodiments, tissue adhesion of the polyurethane can also be increased or decreased by incorporating certain functional groups into the polyurethane (either pendantly or in the polymer backbone) that can non-covalently interact with functional groups in the native tissue matrix, such as through hydrogen bonding and van der Waals interactions. In certain embodiments, the tissue adhesion of the polyurethane can be enhanced by using one or more poly(cyclic carbonates) to synthesize a poly(3-hydroxyurethane), so that the pendant β-hydroxy groups of the polymer can interact with hydrogen bond acceptors in the surrounding native tissue matrix.

In certain embodiments, the resorption profile of the presently disclosed polyurethane-based tissue fillers can be enhanced or decreased by modulation of the amount of functional groups (e.g., ester bonds) in the polyurethane backbone that are hydrolyzable under physiological conditions. Thus, in certain embodiments, the biopersistance of the polyurethane can be controlled by modulating the rate and degree to which the polyurethane is hydrolyzed under physiological conditions. In certain embodiments, the bioresorbability of the polyurethane can be enhanced or decreased by increasing or decreasing the number of ester bonds in the polyurethane backbone. In certain embodiments, the number of ester bonds in the polyurethane backbone can be increased by using a polyether-polyester polyol or a polyester polyol as the polyol in the presently disclosed polyurethane reaction mixture or, alternatively, as the base polymer from which to synthesize a poly(cyclic carbonate), which is then polymerized with at least one polyamine according to the present disclosure.

In certain embodiments, the shape of the three-dimensional structure of the presently disclosed polyurethane-based tissue fillers can be modulated by use of a blowing agent. Thus, in certain embodiments where the polyurethane is polymerized from a polyisocyanate or a polyisocyanate prepolymer, water can be used as a blowing agent. The water reacts with available isocyanate groups to hydrolyze it to the corresponding amino group, releasing gaseous carbon dioxide. In certain other embodiments where the polyurethane is polymerized from a poly(cyclic carbonate), a poly(methylhydrosiloxane) is used as a blowing agent. Available polyamines react with the poly(methylhydrosiloxane) to release nitrogen gas. The gaseous carbon dioxide and nitrogen gas each forms bubbles in the curing polymer, which causes it to expand and develop pores. The rate and degree of expansion of the polymer, as well as the size of the pores, can be modulated by the concentration of blowing agent used. Thus, in certain embodiments, the polyurethane can be engineered to expand to partially or completely fill a void in a tissue, enabling the presently disclosed polyurethane-based tissue fillers to fill irregular space, such as complex/tortuous anal fistulae. In certain embodiments, the presently disclosed polyurethane-based tissue fillers can further comprise a water-binding compound, such as hyaluronic acid, the presence of which will aid in the absorption of water into the pores generated by the foaming of the polyurethane.

In certain embodiments, the above method of treating and/or augmenting tissue in a human or an animal involves filling a void in the tissue of a human or an animal. In certain embodiments, the void in the tissue is the result of damage or loss of tissue due to various diseases and/or structural damage (e.g., from trauma, surgery, atrophy, and/or long-term wear and degeneration). Examples of such voids include, but are not limited to, simple and complex anal fistulae, osteochondral defects (i.e., defects in bone and/or cartilage), tunneling wounds, and other deep wounds to both soft (e.g., muscle) and hard (e.g., bone) tissue. Other possible locations for in vivo delivery of the presently disclosed polyurethane reaction mixtures are into (1) adipose tissue for augmentation procedures, (2) bone or cartilage for orthobiologic applications, or (3) any other soft tissue defect where bulking may be desired. Furthermore, the presently disclosed polyurethane reaction mixtures, as well as the resulting polyurethane-based tissue fillers, can also be used to aesthetically (i.e., cosmetically) augment tissue. Thus, in certain other embodiments, the polyurethane reaction mixture can be injected into the tissue of a human and polymerized to create an aesthetic tissue augmentation implant. Examples of human tissues that can be aesthetically augmented using the presently disclosed compositions include, but are not limited to, breast tissue, buttock tissue, chest tissue, thigh tissue, calf tissue, and facial tissue, including lip and cheek tissue. Examples of particular cosmetic applications for which the presently disclosed precursor compounds, as well as the resulting crosslinked hydrogels, may be used include, but are not limited to, facelift procedures, treatment of facial wrinkles, lines, or other facial features.

In certain embodiments, the polyurethane-based tissue fillers of the present disclosure can include a particulate acellular tissue material that has the biologic ability to support tissue regeneration. In certain embodiments, polyurethane-based tissue fillers can support cell ingrowth and differentiation. For example, the tissue fillers can be used for tissue ingrowth, orthopedic surgery, periodontal applications, tissue remodeling, or tissue restoration. In certain embodiments, the tissue fillers produce a regenerative tissue response, as demonstrated by the presence of fibroblast-like cells and blood vessels.

In certain embodiments, the tissue fillers can be used for treatment of numerous different anatomical sites and can be used in a wide array of applications. Certain exemplary applications include, but are not limited to, dermal regeneration (e.g., for treatments of all types of ulcers and burns), nerve regeneration, cartilage regeneration, connective tissue regeneration or repair, bone regeneration, vascular regeneration, cosmetic surgery, and replacement of lost tissue (e.g., after trauma, breast reduction, mastectomy, lumpectomy, parotidectomy, or excision of tumors).

In some embodiments, the tissue filler elicits a reduced immunological or inflammatory response when implanted in an animal compared to the polyurethane alone. The effect of the tissue filler in the host can be tested using a number of methods. For example, in some embodiments, the effect of the tissue filler in the host can be tested by measuring immunological or inflammatory response to the implanted scaffold. The immunological or inflammatory response to the tissue filler can be measured by a number of methods, including histological methods. For example, explanted filler can be stained and observed under a microscope for histological evaluation, as described further below. In some embodiments, the immunological or inflammatory response to the filler can be demonstrated by measuring the number of inflammatory cells (e.g., leukocytes). The attenuated immunological or inflammatory response to the filler may be associated with a reduced number of inflammatory cells, as described further below. For example, inflammatory cells can be measured through immuno-histochemical staining methods designed to identify lymphocytes, macrophages, and neutrophils. Immuno-histochemical methods may also be used to determine the presence of inflammatory cytokines including interleulin-1, TNF-α, and TGF-β.

The presently disclosed tissue filler materials can be used to treat soft tissues in many different tissue types or organ systems. These organ systems can include, but are not limited to, the muscular system, the genitourinary system, the gastroenterological system, the integumentary system, the circulatory system, and the respiratory system. The tissue fillers can also be useful to treat connective tissue, including the fascia, a specialized layer that surrounds muscles, bones, and joints of the chest and abdominal wall, or for repair and reinforcement of tissue weaknesses in urological, gynecological, or gastroenterological anatomy. In certain embodiments, the tissue or organ in need of treatment can be selected from the group consisting of skin, bone, cartilage, meniscus, dermis, myocardium, periosteum, artery, vein, stomach, small intestine, large intestine, diaphragm, tendon, ligament, neural tissue, striated muscle, smooth muscle, bladder, urethra, ureter, or gingival.

Another aspect of the present invention are kits comprising the presently disclosed compositions. At a minimum, such kits comprise (1) a polyurethane precursor and (2) an acellular tissue matrix, as discussed above. In certain embodiments, the kit further comprises (3) at least one catalyst, (4) one or more crosslinking agents, chain extending agents, blowing agents, surfactants, water-miscible solvents, water-binding compounds, or any combination thereof, and/or a device capable of mixing components (1), (2), (3), and (4) and/or injecting a mixture of components (1), (2), (3), and (4). In certain embodiments, the device is selected from the group consisting of single barrel syringes, dual barrel syringe systems, cannulae, syringe-to-syringe luer lock adapter-based systems, in-line static mixers, mixing tips, or any combination thereof.

From the above discussion, one skilled in the art can ascertain the essential characteristics of this invention, and without departing from the spirit and scope thereof, can make various changes and modifications of the invention to adapt it to various uses and conditions.

1. A composition comprising (1) a polyurethane precursor and (2) a particulate acellular tissue matrix.

2. The composition of claim 1, wherein the polyurethane precursor comprises at least one polyol.

3. The composition of claim 2, wherein the composition further comprises at least one catalyst.

4. The composition of claim 2, wherein the composition further comprises one or more crosslinking agents, chain extending agents, blowing agents, surfactants, water-miscible solvents, water-binding compounds, or any combination thereof.

5. The composition of claim 4, wherein the composition further comprises water.

6. The composition of claim 1, wherein the polyurethane precursor comprises at least one polyamine.

7. The composition of claim 6, wherein the composition further comprises at least one catalyst.

8. The composition of claim 6, wherein the composition further comprises one or more crosslinking agents, chain extending agents, blowing agents, surfactants, water-miscible solvents, water-binding compounds, or any combination thereof.

9. The composition of claim 8, wherein the composition further comprises a poly(methylhydrosiloxane).

10. The composition of claim 2, wherein the composition further comprises at least one polyisocyanate.

11. The composition of claim 10, wherein the at least one polyisocyanate is a diisocyanate selected from the group consisting of toluene diisocyanate (TDI), methylene diphenyl diisocyanate (MDI), 1,6-hexamethylene diisocyanate (HDI), 1-isocyanato-3-isocyanatomethyl-3,5,5-trimethyl-cyclohexane (isophorone diisocyanate, IPDI), 4,4′-diisocyanato dicyclohexylmethane (H₁₂MDI), or combinations thereof.

12. The composition of claim 10, wherein the at least one polyisocyanate is a polyisocyanate prepolymer.

13. The composition of claim 10, wherein the composition further comprises at least one catalyst.

14. The composition of claim 10, wherein the composition further comprises one or more crosslinking agents, chain extending agents, blowing agents, surfactants, water-miscible solvents, water-binding compounds, or any combination thereof.

15. The composition of claim 14, wherein the composition further comprises water.

16. The composition of claim 6, wherein the composition further comprises at least one polycyclic carbonate.

17. The composition of claim 16, wherein the composition further comprises at least one catalyst.

18. The composition of claim 16, wherein the composition further comprises one or more crosslinking agents, chain extending agents, blowing agents, surfactants, water-miscible solvents, water-binding compounds, or any combination thereof.

19. The composition of claim 18, wherein the composition further comprises a poly(methylhydrosiloxane).

20. The composition of claim 6, wherein the composition further comprises at least one polyisocyanate prepolymer.

21. The composition of claim 20, wherein the composition further comprises at least one catalyst.

22. The composition of claim 20, wherein the composition further comprises one or more crosslinking agents, chain extending agents, blowing agents, surfactants, water-miscible solvents, water-binding compounds, or any combination thereof.

23. The composition of claim 22, wherein the composition further comprises water.

24. The composition of claim 1, wherein the particulate acellular tissue matrix is derived from dermal tissue, adipose tissue, muscle tissue, bone tissue, cartilage tissue, or any combination thereof.

25. The composition of claim 1, wherein the particulate acellular tissue matrix used to form the composition is in the form of a slurry, a cryomilled dry powder, or micronized particles.

26. The composition of claim 1, wherein the weight ratio of polyurethane precursor to particulate acellular tissue matrix in the composition is in the range of from 1:9 to 9:9.

27. The composition of claim 1, wherein the composition is in the form of a solution, a suspension, a dispersion, an emulsion, or any combination thereof.

28-67. (canceled)
